(19) United States
(12) Patent Application Publication
(10) Pub. No.: US 2006/0287605 A1
(43) Pub. Date: Dec. 21, 2006

(54) HEART RATE VARIABILITY ANALYZING DEVICE

(75) Inventors: Kang-Ping Lin, Jhongli City (TW); Geng-Hong Lin, Pingtung City (TW); Bor-Iuan Jan, Pingtung City (TW)

Correspondence Address: NIKOLAI & MERSEREAU, P.A., 900 SECOND AVENUE SOUTH, SUITE 820, MINNEAPOLIS, MN, 55402, US

(73) Assignee: DAILYCARE BIOMEDICAL INC., Chung-Li City (TW)

(21) Appl. No.: 11/160,281

(22) Filed: Jun. 16, 2005

(51) Int. Cl. A61B 5/04

(52) U.S. Cl. 600/521

(57) ABSTRACT

The invention discloses an heart rate variability (HRV) analyzing device, which integrates the ECG signal measuring and processing technology and combines the HRV analyzing functions. The device adopts sensing electrodes disposed thereon or external sensing electrodes to record a subject's ECG signals. After being amplified, filtered and analog-to-digital converted, the ECG signals are transmitted to a built-in CPU to proceed with time domain and frequency domain analysis, and the results of the analysis are shown on a display unit. In such a way, the signal acquisition, algorithm calculation and display can be completed in the single device of the invention, which simplifies the conventional instruments and equipment, and provides an indication of the subject's health state in the aspect of self-health care.

### BACKGROUND OF THE INVENTION

1. Field of the Invention

The present invention relates to a physiological signal analyzing device, and more particularly, to a heart rate variability (HRV) analyzing device, which has a heart rate variability analyzing module in a central processor unit (CPU) so as not only to record a person's electrocardiographic signals (ECG signals), but also to calculate parameters related to heart rate variability.

2. Description of Related Art

The autonomic nervous system in a human's body is divided into two parts: the sympathetic nervous system and the parasympathetic nervous system both of which are distributed respectively in the different positions of the human's body and have different functions of interaction and antagonism. When a person accepts stimuli, the effects of the sympathetic nervous system reveal, for example, mydriasis, bronchiectasis, cardioaccelerator, systole enhancing, blood pressure increasing, blood sugar increase and so on. The effects of the parasympathetic nervous system are opposed to the effects of the sympathetic nervous system, but the sympathetic nervous system and the parasympathetic nervous system are generally kept in a balanced situation. If imbalance between the sympathetic nervous system and the parasympathetic nervous system happens, various diseases would be caused in the body. In the day time or when the person's alertness is acute, the activity of the sympathetic nervous system is stronger, while at night or in sleep, the activity of the parasympathetic nervous system is stronger. A healthy person's sympathetic nervous system and parasympathetic nervous system will coordinate with each other according to the body's state. However, if the autonomic nervous system is in disorder, that is, unable to timely coordinate according to the body's state, some bothersome health problems would happen, such as dyspnea, palpitations, intestines and stomach abnormalities, insomnia, etc., and some serious problems, for example, heart disease, hypertension, and even sudden death would be caused.

Research on the autonomic nervous system has proceeded for several years, and the most common way used to investigate the interaction between the sympathetic nervous system and the parasympathetic nervous system is heart rate variability analysis. The heart rate variability is mainly used to explore the relationship between the heart rate interval variation and the physiological mechanism, wherein the heart rate interval refers to a period of time required to generate each heartbeat. Under the standard of heart rate variability signal measurement and analysis set forth by the European Society of Cardiology and the North American Society of Pacing and Electrophysiology in 1996, there are two kinds of heart rate variability analysis: one is time domain analysis, and the other is frequency domain analysis. Time domain analysis that can be calculated by mean heart rate, the mean NN internal (that is, all intervals between adjacent QRS complexes resulting from sinus node depolarizations, so-called normal-to-normal (NN) intervals), the square root of the mean squared differences of successive NN intervals (RMSSD); Standard deviation of the NN intervals (SDSD); etc. Geometrical calculations can be obtained by HRV triangular index measurement, the triangular interpolation of NN interval histogram (TINN), etc.

Frequency domain analysis is to convert the heart rate interval signals varied with time into heart rate interval frequency spectrum, the intensity of which is the square of the sine wave amplitude, and to quantify the relative intensities to obtain power spectral density (PSD). In such a way, even miniscule undulation in the heart rate variability will be apparent. The frequency domain analysis for heart rate variability can be further divided into high-frequency (HF) and low-frequency (LF). The total area under the curve line of power frequency spectrum is called total power (TP), wherein the area in high frequency is called high-frequency power (HFP), and the area in low frequency is called low-frequency power (LFP). The European Society of Cardiology and the North American Society of Pacing and Electrophysiology also defined the high frequency range from 0.15 to 0.4 Hz, and the low frequency range from 0.04 to 0.15 Hz. The high-frequency power is significantly related to the accommodation of the parasympathetic nervous system, while the low-frequency power is related to the adjustment of the sympathetic and parasympathetic nervous systems and the traction of the rennin blood vessel. Since the interaction mechanism of the autonomic nervous system is complex, and the regulating factors are numerous and not easy to be identified, the exact physiological mechanisms still need to be researched and developed in depth. At present, in addition to high frequency and low frequency, research further defines the very low-frequency (VLF), which is ≦0.04 Hz, out of the low frequency. In extended heart rate variability analysis, for example, 12 hours or 24 hours, ultra low-frequency (ULF), which is ≧0.003 Hz, is defined to verify the regulating mechanism of the autonomic nervous system from subtle aspect.

The degree of heart rate variability not only indicates the periodic variation of rhythm of the heart and reflects the autonomic nervous system regulating mechanism, but also, more significantly, displays its high relation with the sudden death potential risk. In recent years, perhaps resulting from the work pressure or environmental factors, sudden death cases have been growing. Medically, the term “sudden death” means that a patient loses life within one hour after suffering an acute cardiac symptom no matter if the patient originally had heart disease. The time and the mode of such death are unforeseen and unheralded. Generally, the cause of sudden death is related to cardiovascular diseases, such as cardiopathy, coronary artery disease, ventricular fibrillation, etc. In statistics, eighty percent of sudden death results from coronary artery diseases and other causes including ventricular hypertrophy, cardiomyopathy, heart failure, myocarditis, valvulopathy, or congenital heart disease.

Recent research reported that heart rate variability is a reference to the potential of sudden death, and according to the study results by Huikuri HV et al. (1992) and Sasaki T. et al. (1999), the decrease of heart rate variability is particularly related to the increase of sudden death. Because the heart rate variability can reflect the actions of the heart and the autonomic nervous regulating mechanism, when the actions of the heart and the autonomic nervous regulating mechanism are normal, indicating the rhythm of the heart being able to timely adjust to the body's state, the heart rate variability is relatively high; while the actions of the heart are unusual or the autonomic nervous regulating mechanism can not timely respond to the body's state, the heart rate variability is relatively low. Thus, although the exact cause of sudden death is not understood, the heart rate variability is a very important referential indicator, as are cardiopathy, coronary artery disease, arrhythmia, ventricular fibrillation, etc. The heart rate variability is more significant to the socio-medical term “karoshi” used in Japan to describe the steadily increasing phenomenon of sudden death resulting from extreme physiological and psychological pressures at work. Research indicates that a person who has spent long time under high stress working conditions has lower heart rate variability than a normal-working person, and has more possibility to suffer sudden death.

Since 1996, though, the European of Cardiology and North American Society of Pacing and Electrophysiology have published reports about heart rate variability measuring and analyzing standards, but in practical application, the electrocardiographic signal (ECG signal) measurement and the heart rate variability analysis are separately carried out. In the market, most relevant heart rate variability analyzing products are in the form of analyzing packages, such as EZ-HRV. To analyze the heart rate variability, the conventional method is to first get a subject's ECG signals, and then transmit the signals to a computer to calculate and obtain the heart rate variability analysis. Although, in today's technology, signal transmission is rapid and convenient, and the auto-signal-transmitting is also not difficult, the ECG measuring still involves the use of electrodes, and after signals have been transmitted to the computer, the HRV analyzing interface still needs to performed, which is not familiar and convenient to ordinary people. Therefore, even if the heart rate variability is a very good indication for assessing a patient's physiological state, including the autonomic nervous system, sudden death risk, etc., such knowledge remains arcane and is mainly applied in the scopes of research, clinical diagnosis, and the like.

Seeing that the heart rate variability is mainly developed as an analyzing package, carried out separately with the acquisition of ECG signals and is not universal to the masses, the present invention provides an HRV analyzing device that gets the subject's ECG signals via internal or external sensing electrodes, and after signal amplifying, filtering and analog-to-digital converting, uses a CPU with an HRV analyzing module therein to analyze time domain and frequency domain HRV parameters, and shows the results on a display unit. By the present invention, the signal acquisition, algorithm calculation and display can be carried out in a single device, not only simplifying the existing instruments, but also letting lay people know their body's state by themselves based on the HRV parameters whereby they can adjust their work and rest.

### SUMMARY OF THE INVENTION

An objective of the present invention is to provide a heart rate variability analyzing device, which includes built-in sensing electrodes and a CPU with a heart rate variability module therein, and is able to instantaneously execute the heart rate variability analysis after recording signals.

It is another objective of the present invention to provide an ECG signal measuring device with an HRV analyzing function, the device that, in addition to getting ECG signals, has a CPU inside to process HRV analysis so as to provide HRV parameters.

It is still another objective of the present invention to provide a physiological alert device to analyze a heart rate by utilizing a built-in HRV analyzing module, so as to let subjects know their body states to enable them to adjust their work and rest.

To attain such objectives, the present invention, integrating micro-electric signal sensing technology with signal processing, utilizes two sensing electrodes to measure signals, and cooperates with a CPU to perform algorithm and analysis for HRV. In such a way, the invention not only records ECG signals, but also provides HRV analyzing parameters. In addition, it is easy to operate the device of the present invention, which completes signals acquisition and HRV analysis at the same time, such that subjects can understand their health states and suitably take care and adjust workload and rest.

Other and further features, advantages and benefits of the invention will become apparent in the following description taken in conjunction with the following drawings. It is to be understood that the foregoing general description and following detailed description are exemplary and explanatory but are not to be restrictive of the invention. The accompanying drawings are incorporated in and constitute a part of this application and, together with the description, serve to explain the principles of the invention in general terms. Like numerals refer to like parts throughout the disclosure.

### BRIEF DESCRIPTION OF THE DRAWINGS

The objects, spirits and advantages of the preferred embodiments of the present invention will be readily understood by the accompanying drawings and detailed descriptions, wherein:

FIG. 1 is a schematic view of ECG signals of normal rhythm of the heart;

FIG. 2 is flow chart of processing HRV analysis;

FIG. 3 is a schematic view of ECG signals of arrhythmia;

FIG. 4 is a block diagram of an embodiment according to the present invention; and

FIG. 5 is a perspective view of the structure of an embodiment according to the present invention.

### DETAILED DESCRIPTION OF THE PREFFERED EMBODIMENT

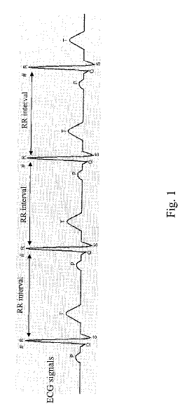

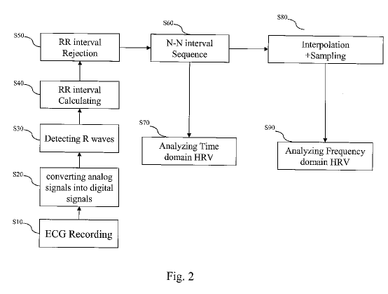

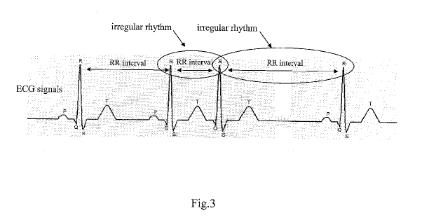

Although the physiological mechanism of the heart rate variability (HRV) has not been understood with certainty so far, the calculation method and process was approximately ascertained in 1996, which was set forth by the European Society of Cardiology and the North American Society of Pacing and Electrophysiology which collected related literatures and published the measuring and analyzing standard of the heart rate variability signals. Since then, the algorithm principles of the HRV analysis in different businesses and products have been almost the same, even though each business has its own software interface and mode of operation, or selects high or low frequency ranges for different objectives. With reference FIG. 1 and FIG. 2, the procedure of the HRV analysis adopted generally and published by the European Society of Cardiology and the North American Society of Pacing and Electrophysiology is explained. In the HRV analysis, the first step (S10) is recording ECG signals, wherein the recording period of ECG signals can be 5 minutes, 12 hours, or 24 hours, etc. according to a subject's need. Clinically, 5 minutes is commonly used as a basic analyzing section, and the recording period of the ECG signals over 5 minutes is considered more meaningful for analyzing the heart rate variability. Then the next step (S20) is converting the ECG signals into digital form, which is followed by step (S30) of detecting R waves. Normally R waves are the maximum peaks in the ECG signals, as indicated as # in the FIG. 1. Since the R waves are obvious, they are usually taken as detecting subject, no matter in heart rate calculation or heart rate analysis. After detecting R waves, the procedure of the heart rate analysis enters step (S40): calculating RR intervals. RR interval is the interval of each heart rate, and after every R wave is marked, RR interval series are formed. Then the next step (50) is rejecting the irregular RR intervals. If the subject has irregular rhythm of the heart, as shown in FIG. 3, the patient will have larger heart rate interval variations than that with regular rhythm of the heart, and the calculated HRV will be also larger than the HRV calculated from one with regular rhythm of the heart, so as not to correctly reflect the subject's health state. Thus the irregular RR intervals need to be weeded out by any mathematical method, such as a standard deviation method, which gets rid of RR intervals exceeding one or three standard deviation of RR interval average, or an average method of averaging the arrhythmia RR interval with the preceding or the next RR interval. After rejecting the irregular RR intervals, the procedure enters step (S60) of getting regular heart rate intervals called as N-N interval sequence. At this time, the N-N interval sequence is processed by time domain HRV analysis, step (S70), for example, computing the mean value, standard deviation, or coefficient of variation, of heart rate intervals; root means square of successive difference; and so on. Alternatively, the procedure can also enter step (S80) of equidistantly sampling with interpolation calculation for frequency domain HRV analysis. Because in step (30), the sampling frequencies of R waves are not all the same, the N-N interval sequence must be converted to continuous signals by interpolation calculation, and equidistantly sampled to proceed to execute step (S90) of executing frequency domain HRV analysis, such as Fourier transform, Hilbert transform, and the like. After converting the signals from time domain to frequency domain, and computing the high-frequency power, low-frequency power and total power, the procedure can obtain frequency domain analyzing parameters, such as HF, LF, LF/HF. LF/TH.

Although the HRV analysis is very useful to assess the regulation mechanism of an autonomic nervous system, the HRV analysis is still not widely applied because HRV analysis is still mainly performed by computer interfacing and the ECG signals recording is complex. In order to make HRV analysis more convenient and quick to serve as an alerting reference for physiological condition, the invention provides an HRV analyzing device, which not only has ECG signals measuring and HRV analyzing functions can provide HRV parameters instantaneously, but also is easily operated.

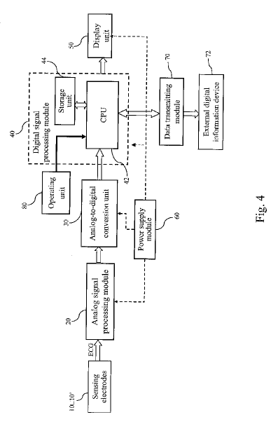

With reference to FIG. 4, the invention includes two sensing electrodes (10, 10′) to contact the subject's body surface, respectively, for recording ECG signals; an analog signal processing module (20) connected to the sensing electrodes (10, 10′) for processing the ECG signals, such as signal amplifying, filtering, etc.; an analog-to-digital conversion unit (30) for converting the analog ECG signals processed by the analog signal processing module (20) to digital ECG signals; a digital signal processing module (40) for processing HRV analysis; a display unit (50) electrically connected to the digital signal processing module (40) for displaying the calculated HRV parameters on a screen, such as an LCD, LED, and the like; and a power supply module (60) electrically connected to all the mentioned-above modules and units for providing power with the device, wherein the power supply module (60) can be a cell set or external power source. A CPU (42) is disposed in the digital signal processing module (40) to process HRV analysis, like step (30) to step (70) and step (30) to step (90) so as to obtain at least one HRV parameter.

The HRV parameters can be the results of time domain analysis or frequency domain analysis. The results of time domain analysis include the mean NN interval, the mean heart rate, standard deviation, coefficient of variation, RMSSD, SDSD . . . and the results of frequency domain analysis include HF, LF, LF/HF, and LF/TP.

In this invention, the CPU (42) of the digital signal processing module (40) is further electrically connected to a storage unit (44) and a data transmission module (70). The storage unit (44) can save the digital ECG signals and HRV parameters. The data transmission module (70) can transmit the data stored in the storage unit (44), including digital ECG signals and HRV parameters, to an external digital information device (72), wherein the data transmission module (70) can use an USB interface, Bluetooth interface, infrared rays interface, modem, etc., and the external digital information device (72) can be a personal computer, PDA, cell phone, database, etc. The HRV analyzing device of the invention further comprises an operating unit (80) electrically connected to the CPU (42) to have the subject be able to control the operation of the digital signal processing module (40). The operating unit (80) can be presented in any manner, such as buttons, knobs, touch panels, etc., to carry out the desired actions, such as performing measuring functions, adding/deleting/transmitting the data in the storage unit (44), inputting the subject's personal information, setting a date, etc.

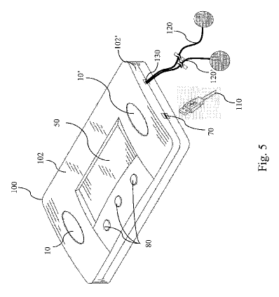

With reference to FIG. 5, which is an embodiment according to the present invention, the HRV analyzing device is a body (100) defining a room therein (not shown in FIG. 5) and having two operation surfaces (102, 102′). The sensing electrodes (10, 10′) are separately disposed on the left and right sides of the operation surface (102). The analog signal processing module (20), the analog-to-digital conversion unit (30) and the digital signal processing module (40), including the CPU (42), and the storage unit (44) are received in the room. The display unit (50) and the operating unit (80) are also disposed on the operation surface (102). The data transmitting module (70) is disposed on the other operation surface (102′).

In the ECG signal measurement principle, when the heart takes systole and diastole, the activity of the myocardial current will be transmitted to the subject's body surface such that, by means of electrodes contacting the body surface, the voltage variations of the heart activity can be recorded. In clinical conditions, the most common way is to use a 12-lead ECG, which includes three standard leads: I, II, III; three augmented leads: aVR, aVL, aVF; and six chest leads: V1, V2, V3, V4, V5, V6. In this invention, the subject just places a left finger and a right finger on the sensing electrodes (10, 10′) and the lead I ECG signals of the subject can be recorded.

In signals processing procedure, the analog signal processing module (20) electrically connected to the sensing electrodes (10, 10′) amplifies and filters the lead I ECG signals, and then transmits the ECG signals to the analog-to-digital conversion unit (30). The analog-to-digital conversion unit (30) converts the analog ECG signals to digital signals, and then transmits the digital signals to the digital signal processing module (40) to analyze HRV. The CPU (42) of the digital signal processing module (40) has an HRV analysis program module to execute the step (S30) to step (S70) or step (S30) to step (S70) with respect to the ECG signals, and obtains the time domain and frequency domain HRV parameters. These HRV parameters and ECG signals can be saved in the storage unit (44) and simultaneously shown on the display unit (20) disposed on the operation surface (102). Accordingly, subjects can know the HRV results of their bodies to attain the alert objectives.

Besides, the HRV analyzing device can be connected to the external information device (72) through the data transmitting module (70) disposed on the operation surface (102′) to transmit the data in the storage unit (44) into the external information device (72). The data transmitting module (70) is not limited to any form, and may be for example, a USB interface, Bluetooth interface, infra rays interface, modem, etc., regardless of wire or wireless manner. On the other hand, all the functions, such as power-on, power-off, setting a date, inputting the subject's personal information, reading/deleting/transmitting the data in the storage unit (44), etc., of the HRV analyzing device can be conveniently operated through the operating unit (80) disposed on the operation surface (102).

There are still two external signal-sensing electrodes (120, 120′) designed to substitute for the sensing electrodes (10, 10′) for measuring ECG signals. As shown in FIG. 5, the external signal-sensing electrodes (120, 120′) are connected to the body (100) via an electrode adaptive port (130) to measure and transmit the ECG signals whenever the subjects are not comfortable enough to stably put their hands on the sensing electrodes (10, 10′). After getting the ECG signals from the external signal-sensing electrodes (120, 120′), the HVR analyzing device also transmits the ECG signals to the analog-to-digital conversion unit (30) to do the same processing as described above. The external signal-sensing electrodes (120, 120′) are used to adhere to the subject's body surface, and can measure lead I, II, III ECG signals according to the vector of adhesive position so as to record different leads ECG signals as the subject needs.

Although this invention has been disclosed and illustrated with reference to particular embodiments, the principles involved are susceptible for use in numerous other embodiments that will be apparent to persons skilled in the art. This invention is, therefore, to be limited only as indicated by the scope of the appended claims.

What is claimed is:

1. A device for analyzing heart rate variability comprising:
a body defining a room therein;
two sensing electrodes disposed on the body for acquiring electrocardiogram signals (ECG signals) of a subject;
an analog signal processing module disposed in the body and electrically connected to the sensing electrodes for analog processing the ECG signals;
an analog-to-digital conversion unit disposed in the body for converting the ECG signals in analog form from the analog signal processing module to the ECG signals in digital form;
a digital signal processing module disposed in the body comprising a center processing unit (CPU) for analyzing the heart rate variability (HRV) in respect of the ECG signals to obtain at least one heart rate variability parameter;
a display unit disposed on the body and electrically connected to the digital signal processing module for showing the heart rate variability parameter; and
a power supply module providing power for all elements described above.

2. The device according to claim 1, wherein the sensing electrodes are in contact with a left finger and a right finger of the subject, respectively, to get the subject's lead I ECG signals.

3. The device according to claim 1, wherein the analog processing module is to amplify and filter the ECG signals.

4. The device according to claim 1, wherein the device executes a procedure of heart rate variability analysis, which comprises steps of:
detecting R waves in the digital ECG signals;
calculating intervals of R waves to get R-R intervals and to form an R-R interval series;
rejecting the irregular R-R intervals having larger variations to obtain N-N interval sequence; and
proceeding statistically a time domain calculation to the N-N interval sequence to obtain time domain HRV parameters.

5. The device according to claim 1, wherein the device executes a procedure of heart rate variability analysis, which comprises steps of:
detecting R waves in the digital ECG signals;
calculating intervals of R waves to get R-R intervals and to form an R-R interval series;
rejecting the irregular R-R intervals having larger variations to obtain N-N interval sequence;
proceeding an interpolation calculation to establish N-N interval continuous signals; and
sampling the N-N interval continuous signals and proceeding a frequency domain calculation to the N-N interval continuous signals to obtain frequency domain HRV parameters.

6. The device according to claim 1, wherein the digital signal processing module further comprises a storage unit connected to the CPU for saving the digital ECG signals and the HRV parameters.

7. The device according to claim 6, wherein the body has a data transmitting module disposed thereon and connected to the CPU for transmitting the ECG signals and the HRV parameters saved in the storage unit to an external digital information device.

8. The device according to claim 7, wherein the data transmission module is a USB transmission interface, a Bluetooth™ transmission interface, an infrared rays transmission interface, or a modem.

9. The device according to claim 7, wherein the external digital information device is a personal computer, a personal digital assistant, a cell phone or database.

10. The device according to claim 1, wherein the CPU is further electrically connected to an operating unit disposed on the body for enabling the subject to set and control the actions of the analyzing device.

11. The device according to claim 1, wherein the analog signal processing module is electrically connected to an electrode adaptive port disposed on the body to connect to external signal sensing electrodes for substituting for the sensing electrodes to get the ECG signals.

12. The device according to claim 1, wherein the power supply module is a cell set disposed in the body.

13. The device according to claim 1, wherein the power supply module is an external power source.

14. A device for analyzing heart rate variability comprising:
two sensing electrodes for acquiring electrocardiographic signals (ECG signals) of a subject;
an analog signal processing module electrically connected to the sensing electrodes for analog processing the ECG signals;
an analog-to-digital conversion unit for converting the ECG signals in analog form from the analog signal processing module to the ECG signals in digital form;
a digital signal processing module comprising a center processing unit (CPU) for analyzing the heart rate variability in respect of the ECG signals to obtain at least one heart rate variability parameter; and
a display unit electrically connected to the digital signal processing module for showing the heart rate variability parameter.

15. The device according to claim 14, wherein the sensing electrodes are in contact with the subject's body surface to get the subject's ECG signals.

16. The device according to claim 14, wherein the analog processing module is to amplify and filter the ECG signals.

17. The device according to claim 14, wherein the device executes a procedure of heart rate variability analysis, which comprises steps of:
detecting R waves in the digital ECG signals;
calculating intervals of R waves to get R-R intervals and to form an R-R interval series;
rejecting the irregular R-R intervals having larger variations to obtain N-N interval sequence; and
proceeding statistically a time domain calculation to the N-N interval sequence to obtain time domain HRV parameters.

18. The device according to claim 14, wherein the device executes a procedure of heart rate variability analysis, which comprises steps of:
detecting R waves in the digital ECG signals;
calculating intervals of R waves to get R-R intervals and to form an R-R interval series;
rejecting the irregular R-R intervals having larger variations to obtain N-N interval sequence;
proceeding an interpolation calculation to establish N-N interval continuous signals; and
sampling the N-N interval continuous signals and proceeding a frequency domain calculation to the N-N interval continuous signals to obtain frequency domain HRV parameters.

19. The device according to claim 14, wherein the digital signal processing module further comprises a storage unit connected to the CPU for saving the digital ECG signals and the HRV parameters.

20. The device according to claim 19, wherein the CPU is electrically connected to a data transmitting module for transmitting the ECG signals and the HRV parameters saved in the storage unit to an external digital information device.

21. The device according to claim 20, wherein the data transmission module is a USB transmission interface, a Bluetooth™ transmission interface, an infrared rays transmission interface, or a modem.

22. The device according to claim 20, wherein the external digital information device is a personal computer, a personal digital assistant, a cell phone or database.

23. The device according to claim 14, wherein the CPU is further electrically connected to an operating unit disposed on the body to enable the subject to set and control the actions of the analyzing device.
